(19) United States
(12) Patent Application Publication
(10) Pub. No.: US 2016/0193295 A1
(43) Pub. Date: Jul. 7, 2016

(54) STABILIZATION OF FC-CONTAINING POLYPEPTIDES

(71) Applicant: AMGEN INC., Thousand Oaks, CA (US)

(72) Inventors: Gunasekaran KANNAN, Newbury Park, CA (US); Jennifer LAVALLEE, Simi Valley, CA (US); Frederick W. JACOBSEN, Newbury Park, CA (US)

(73) Assignee: AMGEN INC., Thousand Oaks, CA (US)

(21) Appl. No.: 14/909,431

(22) PCT Filed: Jul. 30, 2014
(86) PCT No.: PCT/US14/48908

§ 371 Date: Feb. 1, 2016

Related U.S. Application Data
(60) Provisional application No. 61/860,800, filed on Jul. 31, 2013.

(51) Int. Cl. A61K 38/18; A61K 47/48; C07K 14/475

(52) U.S. Cl. CPC A61K 38/18; C07K 14/475; A61K 47/48376; C07K 2319/30

(57) ABSTRACT

This disclosure provides polypeptides comprising an antibody Fc region having a deletion of one or more cysteine residues in the hinge region and substitution with a sulfhydryl-containing residue of one or more CH3-interface amino acids. Also, provided are Fc-fusion proteins and antibodies containing said polypeptides, nucleic acids and vectors encoding said polypeptides, along with host cells and methods for making said polypeptides.

### CROSS-REFERENCE TO RELATED APPLICATIONS

This application claims the benefit of U.S. Provisional Application No. 61/860,800, filed Jul. 31, 2013, which is hereby incorporated by reference.

### SEQUENCE LISTING

The instant application contains a Sequence Listing which has been submitted electronically in ASCII format and is hereby incorporated by reference in its entirety. Said ASCII copy, created on Jul. 28, 2014, is named A-1852-WO-PCT_SEtxt and is 122,988 bytes in size.

### BACKGROUND

Antibodies have become the modality of choice within the biopharma industry because they possess several characteristics that are attractive to those developing therapeutic molecules. Along with the ability to target specific structures or cells, antibodies make its target susceptible to Fc-receptor cell-mediated phagocytosis and killing (Raghavan and Bjorkman 1996). Further, the antibody's ability to interact with neonatal Fc-receptor (FcRn) in a pH dependent manner confers it with extended serum half-life (Ghetie and Ward 2000). This unique feature of antibodies allows extending the half-life of therapeutic protein or peptide in the serum by engineering Fc-fusion molecules.

Antibodies belong to the immunoglobulin class of proteins which includes IgG, IgA, IgE, IgM, and IgD. The most abundant immunoglobulin class in human serum is IgG whose schematic structure is shown in FIG. 1 (Deisenhofer 1981; Huber 1984; Roux 1999). The IgG structure has four chains, two light and two heavy chains; each light chain has two domains and each heavy chain has four domains. The antigen binding site is located in the Fab region (Fragment antigen binding) which contains a variable light (VL) and a variable heavy (VH) chain domain as well as constant light (LC) and constant heavy (CH1) chain domains. The hinge, CH2, and CH3 domain region of the heavy chain is called Fc (Fragment crystallizable). The IgG molecule can be considered as a heterotetramer having two heavy chains that are held together by disulfide bonds (—S—S—) at the hinge region and two light chains. The number of hinge disulfide bonds varies among the immunoglobulin subclasses (Papadea and Check 1989). The FcRn binding site is located in the Fc region of the antibody (Martin, West et al. 2001), and thus the extended serum half-life property of the antibody is retained in the Fc fragment. The Fc region alone can be thought of as a homodimer of heavy chains comprising the hinge, CH2 and CH3 domains.

The Fc region of naturally occurring IgG antibodies is a homodimer and can be expressed and purified as a dimer. As discussed above, the Fc region of the antibody confers serum half-life via FcRn recycling mechanism. Hence, the Fc is used as a fusion partner to extend the serum half-life of therapeutic proteins, peptides (peptibody), and protein domains. However, for some therapeutic applications, it may be necessary to delete the hinge region which removes the covalent link between the two polypeptide chains that form Fc. For example, when an Fc is fused to a protein that contains internal disulfide bonds or free cysteine residues, the hinge disulfides could interfere with the folding and lead to aggregation. However, removing the hinge region eliminates the covalent link between the two polypeptide chains. This could lead to disassociation of the noncovalent interaction between the two Fc chains, either during the manufacturing stage or in-vivo, and lead to the association of the Fc chains with other proteins/molecules.

### SUMMARY

As disclosed herein, by introducing a disulfide bond at the CH3 domain interface, the thermal stability of Fc-containing molecules lacking disulfide bonds within the hinge region can be improved. In addition, the covalent link keeps the two polypeptide chains that form dimer in the Fc structure intact without disassociation in-vitro or in-vivo. As shown in FIG. 3, in certain embodiments, the only covalent link between the two Fc chains in the WT del hinge Fc homodimer and a mutant del hinge Fc heterodimer is the introduced disulfide bond.

In certain embodiments, one or more residues that make up the CH3-CH3 interface on both CH3 domains is replaced with a sulfhydryl containing residue such that the interaction becomes stabilized by the formation of a disulfide bond (—S—S—) between the CH3 domains. In preferred embodiments, an amino acid in the interface, such as a leucine, threonine, serine or tyrosine, is replaced with a cysteine or methionine, preferably cysteine. In certain embodiments, the amino acid is replaced with an unnatural amino acid having the desired charge characteristic, such as homocysteine or glutathione.

In a first aspect of the invention, a polypeptide comprises an antibody Fc region having a deletion or substitution of one or more cysteines of the hinge region and substitution of one or more CH3-interface amino acids with a sulfhydryl containing residue, preferably cysteine. The hinge region may lack cysteine residues by virtue of substitution or through deletion. In certain embodiments, the Fc lacks the hinge region altogether. In other embodiments, only a portion of the hinge region is deleted, preferably a portion comprising the cysteine residues.

In certain embodiments of the first aspect, CH3-interface amino acid Y349, L351, 5354, T394, or Y407 is substituted with a sulfhydryl containing residue, preferably cysteine. In preferred embodiments, the Fc comprises an L351C substitution. When two Fc-containing polypeptides having an L351C substitution interact under appropriate conditions, a disulfide bond is formed between the L351C residues in the two chains. Similarly, when two Fc-containing polypeptides having a T394C substitution interact under appropriate conditions, a disulfide bond is formed between the T394C residues in the two chains. Moreover, when two Fc-containing polypeptides having a Y407C substitution interact under appropriate conditions, a disulfide bond is formed between the Y407C residues in the two chains. When an Fc-containing polypeptide having a Y349C substitution interacts with an Fc-containing polypeptide having an S354C substitution under appropriate conditions, a disulfide bond is formed between the Y349C residue in one chain and the S354C residue in the other chain.

The Fc region of the polypeptide of the first aspect may contain one or more additional amino acid substitutions in the CH2 and/or CH3 region. In preferred embodiments, the Fc region comprises one or more amino acid substitutions in the CH2 region that alter the effector function of an Fc-containing protein as compared to similar protein having a wild-type CH2. In other embodiments, the Fc region comprises one or more amino acid substitutions in the CH3 region that alter the ability of the Fc-containing polypeptide to homodimerize and/or increase the ability to heterodimerize with a Fc-containing polypeptide having reciprocal amino acid substitutions in the CH3 region.

In certain embodiments or the first aspect, one or more amino acids of the C-terminus of the Fc are deleted or substituted. In preferred embodiments, a C-terminal lysine is deleted or substituted for another amino acid. In other embodiments, the two or three terminal amino acids are deleted or substituted for another amino acid.

In certain embodiments of the first aspect, the polypeptide is an antibody heavy chain. In other embodiments, the polypeptide is an Fc-fusion protein. The Fc-fusion protein may contain a linker at the N-terminus and/or C-terminus of the Fc molecule.

In a second aspect of the invention, a nucleic acid encodes a polypeptide of the first aspect.

In a third aspect, an expression vector comprises the nucleic acid of the second aspect operably linked to a regulatory sequence, such as a heterologous promoter and/or enhancer.

In a fourth aspect, a host cell comprises the expression vector of the third aspect. In preferred embodiments, the host cell is a eukaryotic cell, such as a yeast or mammalian cell line. A preferred mammalian cell line is a Chinese hamster ovary (CHO) cell line.

A fifth aspect of the invention is a method of making a polypeptide of the first aspect. The methods comprise culturing a host cell of the fourth aspect under conditions in which the regulatory region is active in the host cell and isolating the polypeptide from the culture.

In a sixth aspect, a pharmaceutic composition comprises a polypeptide of the first aspect.

### BRIEF DESCRIPTION OF THE DRAWINGS

FIG. 1. Schematic diagram of IgG1 antibody with the domains indicated. The IgG1 antibody is a Y-shaped tetramer with two heavy chains (longer length) and two light chains (shorter length). The two heavy chains are linked together by disulfide bonds (—S—S—) at the hinge region. Fab—fragment antigen binding, Fc—fragment crystallizable, VL—variable light chain domain, VH—variable heavy chain domain, CL—constant (no sequence variation) light chain domain, CH1—constant heavy chain domain 1, CH2—constant heavy chain domain 2, CH3—constant heavy chain domain 3.

FIG. 2. Schematics showing Fc dimers, lacking the hinge region (“del hinge”), with an introduced disulfide bond at the CH3 domain interface, (a) in the case of WT Fc homodimer and (b) in the case of mutant Fc heterodimer, in which mutations (e.g., knows-into-holes or charged pair mutations) also have been introduced at the CH3 domain interface.

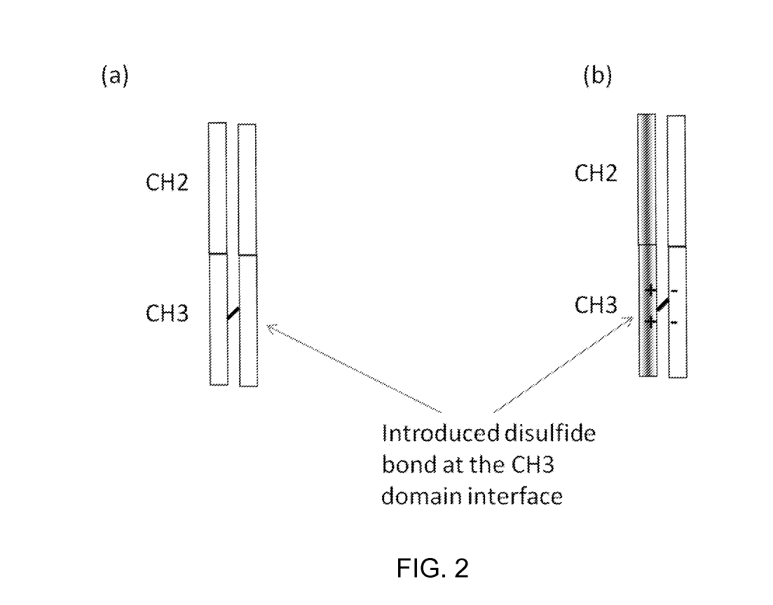

FIG. 3. SDSPAGE showing a major single band confirming the covalent linkage between the positive (‘+’) and negative (‘−’) Fc chains for a del hinge Fc charged pair mutation heterodimer fusion construct with an introduced L351C disulfide bond at the CH3 domain interface.

FIG. 4. Summary of pharmacokinetics of heterodimeric (charge pair mutations) Fc fusion proteins lacking a hinge region. A. Fc fusion lacking hinge and without a linker between the therapeutic peptide and the Fc. B. Same as A except with a variation within the therapeutic peptide. C. Same as B except a non-glycosylated linker connects the Fc to the therapeutic peptide. D. Same as C except with a different linker. E. Same as A except one Fc chain comprises a Y349C substitution and the other comprises an S354C substitution. F. Same as B except one Fc chain comprises a Y349C substitution and the other comprises an S354C substitution.

### DETAILED DESCRIPTION

Described herein are methods of improving stability of antibody Fc scaffolds, particularly Fc scaffolds lacking the hinge region, lacking a portion of the hinge region that forms disulfide bonds, or wherein the hinge region contains substitution of one or more cysteine residues. Such methods involve introducing one or more engineered disulfide bonds at the CH3 domain interface.

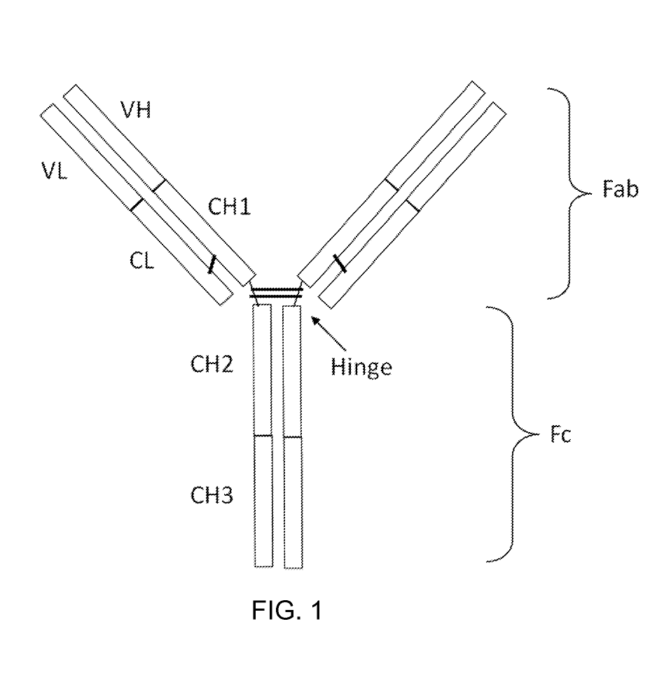

As shown in FIG. 1, the IgG1 antibody is a Y-shaped tetramer with two heavy chains (longer length) and two light chains (shorter length). The two heavy chains are linked together by disulfide bonds (—S—S—) at the hinge region. The IgG molecule can be considered as a heterotetramer consisting of two heavy chains that are held together by disulfide bonds (—S—S—) at the hinge region and two light chains. The number of hinge disulfide bonds varies among the immunoglobulin subclasses.

Covalent linkage between the two heavy chains is provided by the disulfide bonds in the hinge region (which is solvent exposed) in naturally occurring antibodies. Accordingly, in an Fc dimer or antibody lacking the hinge region, there is no covalent linkage between the two heavy chains. The hinge disulfides, together with the disulfide bond between the light and heavy chain (CL-CH1), keep all the four chains covalently linked. The molecular weight of the intact antibody is approximately 150 KDa and runs as a single band in the non-reduced SDSPAGE. There is no disulfide bond at the CH3 domain interface in the WT IgG1/Fc.

An exemplary human IgG1 Fc amino acid sequence is DKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKV SNKALPAPIEKTISKAKGQPREPQVYTLPPSRDELTKNQVSLTCLVKGFYPSDI AVEWESNGQPENNYKTTPPVLDSDGSFFLYSKLTVDKSRWQQGNVFSCSVM HEALHNHYTQKSLSLSPGK (SEQ ID NO: 9)

In the above sequence, DKTHTCPPCPAPELLGG (SEQ ID NO: 10) corresponds to the hinge region.

The amino acids making up the CH3-CH3 interface are described in co-owned provisional application 61/019,569, filed Jan. 7, 2008, and 61/120,305, filed Dec. 5, 2009, along with PCT/US2009/000071, filed Jan. 6, 2009 (all incorporated by reference in their entirety).

A total of 48 antibody crystal structures which had co-ordinates corresponding to the Fc region were identified from the Protein Data Bank (PDB) (Bernstein, Koetzle et al. 1977) using a structure based search algorithm (Ye and Godzik 2004). Examination of the identified Fc crystal structures revealed that the structure determined at highest resolution corresponds to the Fc fragment of RITUXIMAB bound to a minimized version of the B-domain from protein A called Z34C (PDB code: 1L6X). The biological Fc homodimer structure for 1L6X was generated using the deposited Fc monomer co-ordinates and crystal symmetry. Two methods were used to identify the residues involved in the CH3-CH3 domain interaction: (i) contact as determined by distance limit criterion and (ii) solvent accessible surface area analysis.

According to the contact based method, interface residues are defined as residues whose side chain heavy atoms are positioned closer than a specified limit from the heavy atoms of any residues in the second chain. Though 4.5 Å distance limit is preferred, one could also use longer distance limit (for example, 5.5 Å) in order to identify the interface residues (Bahar and Jernigan 1997).

The second method involves calculating solvent accessible surface area (ASA) of the CH3 domain residues in the presence and absence of the second chain (Lee and Richards 1971). The residues that show difference (>1 A²) in ASA between the two calculations are identified as interface residues. Both the methods identified similar set of interface residues. Further, they were consistent with the published work (Miller 1990).

Table 1 lists twenty four interface residues identified based on the contact criterion method, using the distance limit of 4.5 Å. These residues were further examined for structural conservation. For this purpose, 48 Fc crystal structures identified from the PDB were superimposed and analyzed by calculating root mean square deviation for the side chain heavy atoms. The residue designations are based on the EU numbering scheme of Kabat, which also corresponds to the numbering in the Protein Data Bank (PDB).

TABLE 1

| Interface Res in | |
|---|---|
| Chain A | Contacting Residues in Chain B |
| GLN A 347 | LYS B 360′ |
| TYR A 349 | SER B 354′ ASP B 356′ GLU B 357′ LYS B 360′ |
| THR A 350 | SER B 354′ ARG B 355′ |
| LEU A 351 | LEU B 351′ PRO B 352′ PRO B 353′ SER |
| | B 354′ THR B 366′ |
| SER A 354 | TYR B 349′ THR B 350′ LEU B 351′ |
| ARG A 355^{b} | THR B 350′ |
| ASP A 356 | TYR B 349′ LYS B 439′ |
| GLU A 357 | TYR B 349′ LYS B 370′ |
| LYS A 360^{b} | GLN B 347′ TYR B 349′ |
| SER A 364 | LEU B 368′ LYS B 370′ |
| THR A 366 | LEU B 351′ TYR B 407′ |
| LEU A 368 | SER B 364′ LYS B 409′ |
| LYS A 370 | GLU B 357′ SER B 364′ |
| ASN A 390 | SER B 400′ |
| LYS A 392 | LEU B 398′ ASP B 399′ SER B 400′ PHE B 405′ |
| THR A 394 | THR B 394′ VAL B 397′ PHE B 405′ TYR B 407′ |
| PRO A 395 | VAL B 397′ |
| VAL A 397 | THR B 393′ THR B 394′ PRO B 395′ |
| ASP A 399 | LYS B 392′ LYS B 409′ |
| SER A 400 | ASN B 390′ LYS B 392′ |
| PHE A 405 | LYS B 392′ THR B 394′ LYS B 409′ |
| TYR A 407 | THR B 366′ THR B 394′ TYR B 407′ SER B 408′ LYS |
| | B 409′ |
| LYS A 409 | LEU B 368′ ASP B 399′ PHE B 405′ TYR B 407′ |
| LYS A 439 | ASP B 356′ |

The crystal structure of WT Fc was obtained and analyzed for potential positions for the introduction of cysteine residues for an engineered disulfide bond. In particular, positions T394 and L351 were selected. The T394 position of the WT Fc chains is juxtaposed at the CH3 domain interface. Mutating T394 to cysteine on both Fc chains would allow formation of a disulfide bond. Similarly, the L351 position of the WT Fc chains is juxtaposed at the CH3 domain interface. Mutating L351 to cysteine on both Fc chains also would allow formation of a disulfide bond. Mutating both T394 and L351 to cysteine in both Fc chains would allow formation of two disulfide bonds.

Because the disulfide bond involves the same residue in both chains, both the T394 and L351 sites are applicable to WT Fc homodimers as well as engineered Fc heterodimers, such as Fc chains with knobs-into-holes, or charged pair mutations.

Positions Y349 and 5354 are juxtaposed in the WT Fc CH3 interface. In a Fc heterodimer, one CH3 region may contain a Y349C substitution and the other CH3 region may contain a S354C substitution. The stability of charged pair heterodimers comprising the (Y349C/S354C) cysteine clamp mutations was found to be superior to heterodimers that did not comprise the cysteine clamp. In particular, the monomers of a charged pair heterodimers without a cysteine clamp were observed in separate bands on SDS-PAGE, while the charged pair heterodimers with the cysteine clamp mutation were observed in a single band. The same was true of charged pair heterodimers comprising a (L351C/L351C) cysteine clamp mutation.

Heterodimers comprising a first CH3-containing molecule comprising a Y349C substitution and a second CH3-containing molecule comprising a S354C substitution displayed greater stability and higher percentage of heterodimer than those containing wild-type amino acid residues at those positions. Furthermore, heterodimers comprising a first and second CH3-containing molecule, each comprising a L351C substitution displayed greater stability and higher production level than those containing L351.

The interface residues within the CH3 region tend to be highly conserved between the various antibody subclasses, classes, and even between diverse species. Thus, although the embodiments wherein are provided within human IgG1, the cysteine engineering is applicable to other Fc-containing molecules. Exemplary Fc sequences are provided below. The residues corresponding to Y349, L351, S354, T394, or Y407 of human IgG1 within the sequences below may be substituted with a sulfhydryl containing residue, preferably cysteine. The corresponding residues in the other human IgG subclasses are indicated in bold.

>IGHG1_human
(SEQ ID NO: 11)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK
>IGHG2_human
(SEQ ID NO: 12)
RKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKE
YKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSR
WQQGNVFSCSVMHEALHNHYTQKSLSLSPGK
>IGHG3_human
(SEQ ID NO: 13)
LKTPLGDTTHTCPRCPAPELLGGPSVFLFPPKPKDTLMISRTPEVTCVV
VDVSHEDPEVQFKWYVDGVEVHNAKTKPREEQYNSTFRVVSVLTVLHQD
WLNGKEYKCKVSNKALPAPIEKTISKTKGQPREPQVYTLPPSREEMTKN
QVSLTCLVKGFYPSDIAVEWESSGQPENNYNTTPPMLDSDGSFFLYSKL
TVDKSRWQQGNIFSCSVMHEALHNRFTQKSLSLSPGK
>IGHG4_human
(SEQ ID NO: 14)
SKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK
>IGHG1_mu
(SEQ ID NO: 15)
VPRDCGCKPCICTVPEVSSVFIFPPKPKDVLTITLTPKVTCVVVDISKD
DPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTC
MITDFFPEDITVEWQWNGQPAENYKNTQPIMNTNGSYFVYSKLNVQKSN
WEAGNTFTCSVLHEGLHNHHTEKSLSHSPGK
>IGHG2A_mu
(SEQ ID NO: 16)
DKKIEPRGPTIKPCPPCKCPAPNLLGGPSVFIFPPKIKDVLMISLSPIVT
CVVVDVSEDDPDVQISWFVNNVEVHTAQTQTHREDYNSTLRVVSALPIQH
QDWMSGKEFKCKVNNKDLPAPIERTISKPKGSVRAPQVYVLPPPEEEMTK
KQVTLTCMVTDFMPEDIYVEWTNNGKTELNYKNTEPVLDSDGSYFMYSKL
RVEKKNWVERNSYSCSVVHEGLHNHHTTKSFSRTPGK
>IGHG2B_mu
(SEQ ID NO: 17)
EPSGPISTINPCPPCKECHKCPAPNLEGGPSVFIFPPNIKDVLMISLTPK
VTCVVVDVSEDDPDVQISWFVNNVEVHTAQTQTHREDYNSTIRVVSTLPI
QHQDWMSGKEFKCKVNNKDLPSPIERTISKIKGLVRAPQVYTLPPPAEQL
SRKDVSLTCLVVGFNPGDISVEWTSNGHTEENYKDTAPVLDSDGSYFIYS
KLNMKTSKWEKTDSFSCNVRHEGLKNYYLKKTISRSPGK
>IGHG2C_mu
(SEQ ID NO: 18)
EPRVPITQNPCPPLKECPPCAAPDLLGGPSVFIFPPKIKDVLMISLSPMV
TCVVVDVSEDDPDVQISWFVNNVEVHTAQTQTHREDYNSTLRVVSALPIQ
HQDWMSGKEFKCKVNNRALPSPIEKTISKPRGPVRAPQVYVLPPPAEEMT
KKEFSLTCMITGFLPAEIAVDWTSNGRTEQNYKNTATVLDSDGSYFMYSK
LRVQKSTWERGSLFACSVVHEVLHNHLTTKTISRSLGK
>IGHG3_mu
(SEQ ID NO: 19)
EPRIPKPSTPPGSSCPPGNILGGPSVFIFPPKPKDALMISLTPKVTCVVV
DVSEDDPDVHVSWFVDNKEVHTAWTQPREAQYNSTFRVVSALPIQHQDWM
RGKEFKCKVNNKALPAPIERTISKPKGRAQTPQVYTIPPPREQMSKKKVS
LTCLVTNFFSEAISVEWERNGELEQDYKNTPPILDSDGTYFLYSKLTVDT
DSWLQGEIFTCSVVHEALHNHHTQKNLSRSPGK
>IGHG1_sheep
(SEQ ID NO: 20)
EPGCPDPCKHCRCPPPELPGGPSVFIFPPKPKDTLTISGTPEVTCVVVDV
GQDDPEVQFSWFVDNVEVRTARTKPREEQFNSTFRVVSALPIQHQDWTGG
KEFKCKVHNEALPAPIVRTISRTKGQAREPQVYVLAPPQEELSKSTLSVT
CLVTGFYPDYIAVEWQKNGQPESEDKYGTTTSQLDADGSYFLYSRLRVDK
NSWQEGDTYACVVMHEALHNHYTQKSISKPPGK
>IGHG2_sheep
(SEQ ID NO: 21)
GISSDYSKCSKPPCVSRPSVFIFPPKPKDSLMITGTPEVTCVVVDVQGDP
EVQFSWFVDNVEVRTARTKPREEQFNSTFRVVSALPIQHDHWTGGKEFKC
KVHSKGLPAPIVRTISRAKGQAREPQVYVLAPPQEELSKSTLSVTCLVTG
FYPDYIAVEWQRARQPESEDKYGTTTSQLDADGSYFLYSRLRVDKSSWQR
GDTYACVVMHEALHNHYTQKSISKPPGK
>IGHG1_cow
(SEQ ID NO: 22)
DPTCKPSPCDCCPPPELPGGPSVFIFPPKPKDTLTISGTPEVTCVVVDVG
HDDPEVKFSWFVDDVEVNTATTKPREEQFNSTYRVVSALRIQHQDWTGGK
EFKCKVHNEGLPAPIVRTISRTKGPAREPQVYVLAPPQEELSKSTVSLTC
MVTSFYPDYIAVEWQRNGQPESEDKYGTTPPQLDADSSYFLYSKLRVDRN
SWQEGDTYTCVVMHEALHNHYTQKSTSKSAGK
>IGHG2_cow
(SEQ ID NO: 23)
GVSSDCSKPNNQHCCVREPSVFIFPPKPKDTLMITGTPEVTCVVVNVGHD
NPEVQFSWFVDDVEVHTARTKPREEQFNSTYRVVSALPIQHQDWTGGKEF
KCKVNIKGLSASIVRIISRSKGPAREPQVYVLDPPKEELSKSTVSVTCMV
IGFYPEDVDVEWQRDRQTESEDKYRTTPPQLDADRSYFLYSKLRVDRNSW
QRGDTYTCVVMHEALHNHYMQKSTSKSAGK
>IGHG3_cow (3)
(SEQ ID NO: 24)
KSEVEKTPCQCSKCPEPLGGLSVFIFPPKPKDTLTISGTPEVTCVVVDVG
QDDPEVQFSWFVDDVEVHTARTKPREEQFNSTYRVVSALRIQHQDWLQGK
EFKCKVNNKGLPAPIVRTISRTKGQAREPQVYVLAPPREELSKSTLSLTC
LITGFYPEEIDVEWQRNGQPESEDKYHTTAPQLDADGSYFLYSKLRVNKS
SWQEGDHYTCAVMHEALRNHYKEKSISRSPGK
>IGHG1_rat
(SEQ ID NO: 25)
VPRNCGGDCKPCICTGSEVSSVFIFPPKPKDVLTITLTPKVTCVVVDISQ
DDPEVHFSWFVDDVEVHTAQTRPPEEQFNSTFRSVSELPILHQDWLNGRT
FRCKVTSAAFPSPIEKTISKPEGRTQVPHVYTMSPTKEEMTQNEVSITCM
VKGFYPPDIYVEWQMNGQPQENYKNTPPTMDTDGSYFLYSKLNVKKEKWQ
QGNTFTCSVLHEGLHNHHTEKSLSHSPGK
>IGHG2A_rat
(SEQ ID NO: 26)
VPRECNPCGCTGSEVSSVFIFPPKTKDVLTITLTPKVTCVVVDISQNDPE
VRFSWFIDDVEVHTAQTHAPEKQSNSTLRSVSELPIVHRDWLNGKTFKCK
VNSGAFPAPIEKSISKPEGTPRGPQVYTMAPPKEEMTQSQVSITCMVKGF
YPPDIYTEWKMNGQPQENYKNTPPTMDTDGSYFLYSKLNVKKETWQQGNT
FTCSVLHEGLHNHHTEKSLSHSPGK
>IGHG2B_rat
(SEQ ID NO: 27)
ERRNGGIGHKCPTCPTCHKCPVPELLGGPSVFIFPPKPKDILLISQNAKV
TCVVVDVSEEEPDVQFSWFVNNVEVHTAQTQPREEQYNSTFRVVSALPIQ
HQDWMSGKEFKCKVNNKALPSPIEKTISKPKGLVRKPQVYVMGPPTEQLT
EQTVSLTCLTSGFLPNDIGVEWTSNGHIEKNYKNTEPVMDSDGSFFMYSK
LNVERSRWDSRAPFVCSVVHEGLHNHHVEKSISRPPGK
>IGHG_rabbit
(SEQ ID NO: 28)
APSTCSKPTCPPPELLGGPSVFIFPPKPKDTLMISRTPEVTCVVVDVSED
DPEVQFTWYINNEQVRTARPPLREQQFNSTIRVVSTLPIAHEDWLRGKEF
KCKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQR
GDVFTCSVMHEALHNHYTQKSISRSPGK
>IGHG1_horse
(SEQ ID NO: 29)
EPIPDNHQKVCDMSKCPKCPAPELLGGPSVFIFPPNPKDTLMITRTPEVT
CVVVDVSQENPDVKFNWYMDGVEVRTATTRPKEEQFNSTYRVVSVLRIQH
QDWLSGKEFKCKVNNQALPQPIERTITKTKGRSQEPQVYVLAPHPDEDSK
SKVSVTCLVKDFYPPEINIEWQSNGQPELETKYSTTQAQQDSDGSYFLYS
KLSVDRNRWQQGTTFTCGVMHEALHNHYTQKNVSKNPGK
>IGHG2_horse
(SEQ ID NO: 30)
ARVTPVCSLCRGRYPHPIGGPSVFIFPPNPKDALMISRTPVVTCVVVNLS
DQYPDVQFSWYVDNTEVHSAITKQREAQFNSTYRVVSVLPIQHQDWLSGK
EFKCSVTNVGVPQPISRAISRGKGPSRVPQVYVLPPHPDELAKSKVSVTC
LVKDFYPPDISVEWQSNRWPELEGKYSTTPAQLDGDGSYFLYSKLSLETS
RWQQVESFTCAVMHEALHNHFTKTDISESLGK
>IGHG3_horse
(SEQ ID NO: 31)
EPVLPKPTTPAPTVPLTTTVPVETTTPPCPCECPKCPAPELLGGPSVFIF
PPKPKDVLMITRTPEVTCLVVDVSHDSSDVLFTWYVDGTEVKTAKTMPNE
EQNNSTYRVVSVLRIQHQDWLNGKKFKCKVNNQALPAPVERTISKATGQT
RVPQVYVLAPHPDELSKNKVSVTCLVKDFLPTDITVEWQSNEHPEPEGKY
RTTEAQKDSDGSYFLYSKLTVETDRWQQGTTFTCVVMHEALHNHVMQKNV
SHSPGK
>IGHG4_horse
(SEQ ID NO: 32)
VIKECGGCPTCPECLSVGPSVFIFPPKPKDVLMISRTPTVTCVVVDVGHD
FPDVQFNWYVDGVETHTATTEPKQEQNNSTYRVVSILAIQHKDWLSGKEF
KCKVNNQALPAPVQKTISKPTGQPREPQVYVLAPHRAELSKNKVSVTCLV
KDFYPTDIDIEWKSNGQPEPETKYSTTPAQLDSDGSYFLYSKLTVETNRW
QQGTTFTCAVMHEALHNHYTEKSVSKSPGK
>IGHG5_horse
(SEQ ID NO: 33)
VVKGSPCPKCPAPELPGGPSVFIFPPKPKDVLKISRKPEVTCVVVDLGHD
DPDVQFTWFVDGVETHTATTEPKEEQFNSTYRVVSVLPIQHQDWLSGKEF
KCSVTNKALPAPVERTTSKAKGQLRVPQVYVLAPHPDELAKNTVSVTCLV
KDFYPPEIDVEWQSNEHPEPEGKYSTTPAQLNSDGSYFLYSKLSVETSRW
KQGESFTCGVMHEAVENHYTQKNVSHSPGK
>IGHG6_horse
(SEQ ID NO: 34)
VIKEPCCCPKCPDSKFLGRPSVFIFPPNPKDTLMISRTPEVTCVVVDVSQ
ENPDVKFNWYVDGVEAHTATTKAKEKQDNSTYRVVSVLPIQHQDWRRGKE
FKCKVNNRALPAPVERTITKAKGELQDPKVYILAPHREEVTKNTVSVTCL
VKDFYPPDINVEWQSNEEPEPEVKYSTTPAQLDGDGSYFLYSKLTVETDR
WEQGESFTCVVMHEAIRHTYRQKSITNFPGK
>Macfas_IGHG1
(SEQ ID NO: 35)
EIKTCGGGSKPPTCPPCPAPELLGGPSVFLFPPKPKDTLMISRTPEVTCV
VVDVSQEDPDVKFNWYVNGAEVHHAQTKPRETQYNSTYRVVSVLTVTHQD
WLNGKEYTCKVSNKALPAPIQKTISKDKGQPREPQVYTLPPSREELTKNQ
VSLTCLVKGFYPSDIVVEWESSGQPENTYKTTPPVLDSDGSYFLYSKLTV
DKSRWRQGNVFSCSVMHEALHNHYTQKSLSLSPGK
>Macmul_IGHG1
(SEQ ID NO: 36)
EIKTCGGGSKPPTCPPCTSPELLGGPSVFLFPPKPKDTLMISRTPEVTCV
VVDVSQEDPDVKFNWYVNGAEVHHAQTKPRETQYNSTYRVVSVLTVTHQD
WLNGKEYTCKVSNKALPAPIQKTISKDKGQPREPQVYTLPPSREELTKNQ
VSLTCLVKGFYPSDIVVEWESSGQPENTYKTTPPVLDSDGSYFLYSKLTV
DKSRWQQGNVFSCSVMHEALHNHYTQKSLSLSPGK
>Macmul_IGHG2
(SEQ ID NO: 37)
GLPCRSTCPPCPAELLGGPSVFLFPPKPKDTLMISRTPEVTCVVVDVSQE
EPDVKFNWYVDGVEVHNAQTKPREEQFNSTYRVVSVLTVTHQDWLNGKEY
TCKVSNKALPAPKQKTVSKTKGQPREPQVYTLPPPRKELTKNQVSLTCLV
KGFYPSDIVVEWASNGQPENTYKTTPPVLDSDGSYFLYSKLTVDKSRWQQ
GNTFSCSVMHEALHNHYTQKSLSLSPGK
>Macmul_IGHG3
(SEQ ID NO: 38)
EFTPPCGDTTPPCPPCPAPELLGGPSVFLFPPKPKDTLMISRTPEVTCVV
VDVSQEDPEVQFNWYVDGAEVHHAQTKPREEQFNSTYRVVSVLTVTHQDW
LNGKEYTCKVSNKGLPAPIEKTISKAKGQPREPQVYILPPPQEELTKNQV
SLTCLVTGFYPSDIAVEWESNGQPENTYKTTPPVLDSDGSYFLYSKLTVD
KSRWQQGNTFSCSVMHEALHNHYTQKSLSVSP
>IGHG1_pig
(SEQ ID NO: 39)
GIHQPQTCPICPGCEVAGPSVFIFPPKPKDTLMISQTPEVTCVVVDVSKE
HAEVQFSWYVDGVEVHTAETRPKEEQFNSTYRVVSVLPIQHQDWLKGKEF
KCKVNNVDLPAPITRTISKAIGQSREPQVYTLPPPAEELSRSKVTLTCLV
IGFYPPDIHVEWKSNGQPEPENTYRTTPPQQDVDGTFFLYSKLAVDKARW
DHGDKFECAVMHEALHNHYTQKSISKTQGK
>IGHG2A_pig
(SEQ ID NO: 40)
GTKTKPPCPICPACESPGPSVFIFPPKPKDTLMISRTPQVTCVVVDVSQE
NPEVQFSWYVDGVEVHTAQTRPKEEQFNSTYRVVSVLPIQHQDWLNGKEF
KCKVNNKDLPAPITRIISKAKGQTREPQVYTLPPHAEELSRSKVSITCLV
IGFYPPDIDVEWQRNGQPEPEGNYRTTPPQQDVDGTYFLYSKFSVDKASW
QGGGIFQCAVMHEALHNHYTQKSISKTPGK
>IGHG2B_pig
(SEQ ID NO: 41)
GTKTKPPCPICPACESPGPSVFIFPPKPKDTLMISRTPQVTCVVVDVSQ
ENPEVQFSWYVDGVEVHTAQTRPKEEQFNSTYRVVSVLPIQHQDWLNGK
EFKCKVNNKDLPAPITRIISKAKGQTREPQVYTLPPHAEELSRSKVSIT
CLVIGFYPPDIDVEWQRNGQPEPEGNYRTTPPQQDVDGTYFLYSKFSVD
KASWQGGGIFQCAVMHEALHNHYTQKSISKTPGK
>IGHG3_pig
(SEQ ID NO: 42)
GTKTKPPCPICPGCEVAGPSVFIFPPKPKDTLMISQTPEVTCVVVDVSK
EHAEVQFSWYVDGVEVHTAETRPKEEQFNSTYRVVSVLPIQHQDWLKGK
EFKCKVNNVDLPAPITRTISKAIGQSREPQVYTLPPPAEELSRSKVTVT
CLVIGFYPPDIHVEWKSNGQPEPEGNYRTTPPQQDVDGTFFLYSKLAVD
KARWDHGETFECAVMHEALHNHYTQKSISKTQGK
>IGHG4_pig
(SEQ ID NO: 43)
GTKTKPPCPICPACEGPGPSAFIFPPKPKDTLMISRTPKVTCVVVDVSQ
ENPEVQFSWYVDGVEVHTAQTRPKEEQFNSTYRVVSVLPIQHQDWLNGK
EFKCKVNNKDLPAPITRIISKAKGQTREPQVYTLPPPTEELSRSKVTLT
CLVTGFYPPDIDVEWQRNGQPEPEGNYRTTPPQQDVDGTYFLYSKLAVD
KASWQRGDTFQCAVMHEALHNHYTQKSIFKTPGK
>IGHG5_pig
(SEQ ID NO: 44)
GRPCPICPACEGPGPSAFIFPPKPKDTEMISRTPKVTCVVVDVSQENPE
VQFSWYVDGVEVHTAQTRPKEEQFNSTYRVVSVLPIQHQDWLNGKEFKC
KVNNKDLPAPITRIISKAKGQTREPQVYTLPPPTEELSRSKLSVTCLIT
GFYPPDIDVEWQRNGQPEPEGNYRTTPPQQDVDGTYFLYSKLAVDKASW
QRGDPFQCAVMHEALHNHYTQKSIFKTPGN

In certain embodiments, the polypeptide containing the CH3 region is an IgG molecule and further contains a CH1 and CH2 domain. Exemplary human IgG sequences comprise the constant regions of IgG1 (e.g., SEQ ID NO:1), IgG2 (e.g., SEQ ID NO:2), IgG3 (e.g., SEQ ID NO:3), and IgG4 (e.g., SEQ ID NO:4).

The Fc region also may be comprised within or derived from the constant region of an IgA (e.g., SEQ ID NO:5), IgD (e.g., SEQ ID NO:6), IgE (e.g., SEQ ID NO:7), and IgM (e.g., SEQ ID NO: 8) heavy chain.

Preferred embodiments of the invention include but are not limited to an antibody, a bispecific antibody, a monospecific monovalent antibody, a bispecific maxibody (maxibody refers to scFv-Fc), a monobody, a peptibody, a bispecific peptibody, a monovalent peptibody (a peptide fused to one arm of a heterodimeric Fc molecule), and a receptor-Fc fusion protein.

In some embodiments, this strategy may be used alongside other strategies for altering interactions of the antibody domains, e.g., altering a CH3 domain to reduce or to favor the ability of the domain to interact with itself.

When the replacements are coordinated properly, the charges are favorable for the formation of a disulfide bond between the residues in the interface, which stabilizes heterodimerization formation.

In certain aspects, the invention provides a method of preparing a heterodimeric protein. The heterodimer may comprise a first CH3-containing polypeptide and a second CH3-containing polypeptide that meet together to form an interface engineered to promote and stabilize heterodimer formation. The first CH3-containing polypeptide and second CH3-containing polypeptide are engineered to comprise one or more sulfhydryl-containing amino acids within the interface that are located to allow formation of a disulfide bond between the sulfhydryl group of an amino acid on the first CH3-containing heterodimer and a sulfhydryl group of an amino acid on the second CH3-containing heterodimer.

In certain embodiments, the CH3-containing polypeptide comprises an IgG Fc region, preferably derived from a wild-type human IgG Fc region. By “wild-type” human IgG Fc it is meant a sequence of amino acids that occurs naturally within the human population. Of course, just as the Fc sequence may vary slightly between individuals, one or more alterations may be made to a wild-type sequence and still remain within the scope of the invention. For example, the Fc region may contain additional alterations that are not related to the present invention, such as a mutation in a glycosylation site, inclusion of an unnatural amino acid or “knobs-into-holes” or “charged pair” mutations.

Additional mutations that may be made to the IgG1 Fc include those facilitate heterodimer formation amongst Fc-containing polypeptides. In some embodiments, Fc region is engineering to create “knobs” and “holes” which facilitate heterodimer formation of two different Fc-containing polypeptide chains when co-expressed in a cell. U.S. Pat. No. 7,695,963. In other embodiments, the Fc region is altered to use electrostatic steering to encourage heterodimer formation while discouraging homodimer formation of two different Fc-containing polypeptide when co-expressed in a cell. WO 09/089,004, which is incorporated herein by reference in its entirety. Preferred heterodimeric Fc include those wherein one chain of the Fc comprises D399K and E356K substitutions and the other chain of the Fc comprises K409D and K392D substitutions. In other embodiments, one chain of the Fc comprises D399K, E356K, and E357K substitutions and the other chain of the Fc comprises K409D, K392D, and K370D substitutions.

The heavy chains may further comprise one of more mutations that affect binding of the antibody containing the heavy chains to one or more Fc receptors. One of the functions of the Fc portion of an antibody is to communicate to the immune system when the antibody binds its target. This is considered “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc to Fc receptors on the surface of cells of the immune system. CDC is mediated through the binding of the Fc with proteins of the complement system, e.g., C1q.

The IgG subclasses vary in their ability to mediate effector functions. For example, IgG1 is much superior to IgG2 and IgG4 at mediating ADCC and CDC. The effector function of an antibody can be increased, or decreased, by introducing one or more mutations into the Fc. Embodiments of the invention include Fc-containing proteins, e.g., antibodies or Fe-fusion proteins, having an Fc engineered to increase effector function (U.S. Pat. No. 7,317,091 and Strohl, Curr. Opin. Biotech., 20:685-691, 2009; both incorporated herein by reference in its entirety). Exemplary IgG1 Fc molecules having increased effector function include (all based on the Eu numbering scheme) those have the following substitutions:
S239D/I332E
S239D/A330S/1332E
S239D/A330L/1332E
S298A/D333A/K334A
P247I/A339D
P247I/A339Q
D280H/K290S
D280H/K290S/S298D
D280H/K290S/S298V
F243L/R292P/Y300L
F243L/R292P/Y300L/P396L
F243L/R292P/Y300L/V305I/P396L
G236A/S239D/I332E
K326A/E333A
K326W/E333S
K290E/S298G/T299A
K290N/S298G/T299A
K290E/S298G/T299A/K326E
K290N/S298G/T299A/K326E
K334V
L235S+S239D+K334V
Q311M+K334V
S239D+K334V
F243V+K334V
E294L+K334V
S298T+K334V
E233L+Q311M+K334V
L234I+Q311M+K334V
S298T+K334V
A330M+K334V
A330F+K334V
Q311M+A330M+K334V
Q311M+A330F+K334V
S298T+A330M+K334V
S298T+A330F+K334V
S239D+A330M+K334V
S239D+S298T+K334V
L234Y+K290Y+Y296W
L234Y+F243V+Y296W
L234Y+E294L+Y296W
L234Y+Y296W
K290Y+Y296W

Further embodiments of the invention include Fc-containing proteins, e.g., antibodies or Fc-fusion proteins, having an Fc engineered to decrease effector function. Exemplary Fc molecules having decreased effector function include (based on the Eu numbering scheme) those have the following substitutions:
N297A or N297Q (IgG1)
L234A/L235A (IgG1)
V234A/G237A (IgG2)
L235A/G237A/E318A (IgG4)
H268Q/V309L/A330S/A331S (IgG2)
C220S/C226S/C229S/P238S (IgG1)
C226S/C229S/E233P/L234V/L235A (IgG1)
L234F/L235E/P331S (IgG1)
S267E/L328F (IgG1)

Another method of increasing effector function of IgG Fc-containing proteins is by reducing the fucosylation of the Fc. Removal of the core fucose from the biantennary complex-type oligosaccharides attached to the Fc greatly increased ADCC effector function without altering antigen binding or CDC effector function. Several ways are known for reducing or abolishing fucosylation of Fc-containing molecules, e.g., antibodies. These include recombinant expression in certain mammalian cell lines including a FUT8 knockout cell line, variant CHO line Lec13, rat hybridoma cell line YB2/0, a cell line comprising a small interfering RNA specifically against the FUT8 gene, and a cell line coexpressing β-1,4-N-acetylglucosaminyltransferase III and Golgi α-mannosidase II. Alternatively, the Fc-containing molecule may be expressed in a non-mammalian cell such as a plant cell, yeast, or prokaryotic cell, e.g., E. coli. Thus, in certain embodiments of the invention, a composition comprises an Fc having reduced fucosylation or lacking fucosylation altogether.

It is known that human IgG1 has a glycosylation site at N297 (EU numbering system) and glycosylation contributes to the effector function of IgG1 antibodies. Groups have mutated N297 in an effort to make aglycosylated antibodies. The mutations have focuses on substituting N297 with amino acids that resemble asparagine in physiochemical nature such as glutamine (N297Q) or with alanine (N297A) which mimics asparagines without polar groups.

As used herein, “aglycosylated antibody” or “aglycosylated fc” refers to the glycosylation status of the residue at position 297 of the Fc. An antibody or other molecule may contain glycosylation at one or more other locations but may still be considered an aglycosylated antibody or aglcosylated Fc-fusion protein.

Co-owned U.S. Provisional Appl. Ser. No. 61/784,669, filed Mar. 14, 2013, describes an effector functionless IgG1 Fc, which is incorporated herein by reference in its entirety. Mutation of amino acid N297 of human IgG1 to glycine, i.e., N297G, provides far superior purification efficiency and biophysical properties over other amino acid substitutions at that residue. Thus, in preferred embodiments, the antibody or Fc-fusion protein comprises a human IgG1 Fc having a N297G substitution.

Aglycosylated IgG1 Fc-containing molecules were shown to be less stable than glycosylated IgG1 Fc-containing molecules. The Fc region may be further engineered to increase the stability of the aglycosylated molecule. In some embodiments, one or more amino acids are substituted to cysteine so to form disulfide bonds in the dimeric state. Residues V259, A287, R292, V302, L306, V323, or 1332 of the CH2 region may be substituted with cysteine. In preferred embodiments, specific pairs of residues are substitution such that they preferentially form a di-sulfide bond with each other, thus limiting or preventing di-sulfide bond scrambling. Preferred pairs include, but are not limited to, A287C and L306C, V259C and L306C, R292C and V302C, and V323C and I332C.

Provided herein are Fc-containing molecules wherein one or more of residues V259, A287, 8292, V302, L306, V323, or 1332 are substituted with cysteine. Preferred Fc-containing molecules include those comprising A287C and L306C, V259C and L306C, R292C and V302C, or V323C and I332C substitutions.

A polypeptide of interest may be fused to the N-terminus or the C-terminus of the IgG Fc region to create an Fe-fusion protein. In certain embodiments, the Fc-fusion protein comprises a linker between the Fc and the polypeptide of interest. Many different linker polypeptides are known in the art and may be used in the context of an Fc-fusion protein. In preferred embodiments, the Fe-fusion protein comprises one or more copies of a peptide consisting of GGGGS (SEQ ID NO: 45), GGNGT (SEQ ID NO: 46), or YGNGT (SEQ ID NO: 47) between the Fc and the peptide or polypeptide of interest. In some embodiments, the polypeptide region between the Fc region and the peptide or polypeptide of interest region comprises a single copy of GGGGS (SEQ ID NO: 45), GGNGT (SEQ ID NO: 46), or YGNGT (SEQ ID NO: 47). The linkers GGNGT (SEQ ID NO: 46) or YGNGT (SEQ ID NO: 47) are glycosylated when expressed in the appropriate cells and such glycosylation may help stabilize the protein in solution and/or when administered in vivo. Thus, in certain embodiments, an Fc fusion protein comprises a glycosylated linker between the Fc region and the protein of interest region.

Co-owned U.S. Provisional Patent Appl. 61/591,161, filed Jan. 26, 2012, and PCT Appl. No. PCT/US2013/023456, filed Jan. 28, 2013, (both incorporated herein by reference in their entirety) describe GDF15 Fc fusion proteins. In certain embodiments of the present invention, a polypeptide comprises an antibody Fc region having a deletion or substitution of one or more cysteines of the hinge region and substitution of one or more CH3-interface amino acids with a sulfhydryl containing residue, preferably cysteine, wherein the polypeptide in not a GDF15 Fc fusion. More specifically, a polypeptide comprises an antibody Fc region having a deletion or substitution of one or more cysteines of the hinge region and substitution of one or more CH3-interface amino acids with a sulfhydryl containing residue, preferably cysteine, wherein the polypeptide in not a GDF15 Fc fusion protein as set forth in PCT Appl. No. PCT/US2013/023456, such as GDF15 fusion proteins comprising:

(SEQ ID NO: 48)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTCPPSRKEMTKNQVSLTCLVKGFYPSDIA
VEWESNGQPENNYKTTPPVLKSDGSFFLYSKLTVDKSRWQQGNVFSCSVM
HEALHNHYTQKSLSLSPG;
(SEQ ID NO: 49)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTCPPSREEMTKNQVSLTCLVKGFYPSDIA
VEWESNGQPENNYDTTPPVLDSDGSFFLYSDLTVDKSRWQQGNVFSCSVM
HEALHNHYTQKSLSLSPGK;
(SEQ ID NO: 50)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTCPPSRKEMTKNQVSLTCLVKGFYPSDIA
VEWESNGQPENNYKTTPPVLKSDGSFFLYSKLTVDKSRWQQGNVFSCSVM
HEALHNHYTQKSLSLSPGGGGGARNGDHCPLGPGRCCRLHTVRASLEDLG
WADWVLSPREVQVTMCIGACPSQFRAANMHAQIKTSLHRLKPDTVPAPCC
VPASYNPMVLIQKTDTGVSLQTYDDLLAKDCHCI;
(SEQ ID NO: 51)
MEWSWVFLFFLSVTTGVHSAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTCPPSRKEMTK
NQVSLTCLVKGFYPSDIAVEWESNGQPENNYKTTPPVLKSDGSFFLYSKL
TVDKSRWQQGNVFSCSVMHEALHNHYTQKSLSLSPGGGGGARNGDHCPLG
RPGRCCLHTVRASLEDLGWADWVLSPREVQVTMCIGACPSQFRAANMHAQ
IKTSLHRLKPDTVPAPCCVPASYNPMVLIQKTDTGVSLQTYDDLLAKDCH
CI;
(SEQ ID NO: 52)
MEWSWVFLFFLSVTTGVHSAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTCPPSREEMTK
NQVSLTCLVKGFYPSDIAVEWESNGQPENNYDTTPPVLDSDGSFFLYSDL
TVDKSRWQQGNVFSCSVMHEALHNHYTQKSLSLSPGK;
(SEQ ID NO: 53)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPCRKEMTKNQVSLTCLVKGFYPSDIA
VEWESNGQPENNYKTTPPVLKSDGSFFLYSKLTVDKSRWQQGNVFSCSVM
HEALHNHYTQKSLSLSPG;
(SEQ ID NO: 54)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVCTLPPSREEMTKNQVSLTCLVKGFYPSDIA
VEWESNGQPENNYDTTPPVLDSDGSFFLYSDLTVDKSRWQQGNVFSCSVM
HEALHNHYTQKSLSLSPGK;
(SEQ ID NO: 55)
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPCRKEMTKNQVSLTCLVKGFYPSDIA
VEWESNGQPENNYKTTPPVLKSDGSFFLYSKLTVDKSRWQQGNVFSCSVM
HEALHNHYTQKSLSLSPGGGGGARNGDHCPLGPGRCCRLHTVRASLEDLG
WADWVLSPREVQVTMCIGACPSQFRAANMHAQIKTSLHRLKPDTVPAPCC
VPASYNPMVLIQKTDTGVSLQTYDDLLAKDCHCI;
(SEQ ID NO: 56)
MEWSWVFLFFLSVTTGVHSAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPCRKEMTK
NQVSLTCLVKGFYPSDIAVEWESNGQPENNYKTTPPVLKSDGSFFLYSKL
TVDKSRWQQGNVFSCSVMHEALHNHYTQKSLSLSPGGGGGARNGDHCPLG
PGRCCRLHTVRASLEDLGWADWVLSPREVQVTMCIGACPSQFRAANMHAQ
IKTSLHRLKPDTVPAPCCVPASYNPMVLIQKTDTGVSLQTYDDLLAKDCH
CI;
or
(SEQ ID NO: 57)
MEWSWVFLFFLSVTTGVHSAPELLGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVCTLPPSREEMTK
NQVSLTCLVKGFYPSDIAVEWESNGQPENNYDTTPPVLDSDGSFFLYSDL
TVDKSRWQQGNVFSCSVMHEALHNHYTQKSLSLSPGK.

Polynucleotides Encoding Antibodies and Fc-Fusion Proteins

Encompassed within the invention are nucleic acids encoding antibody heavy chains and Fc-fusion proteins. Aspects of the invention include polynucleotide variants (e.g., due to degeneracy) that encode the amino acid sequences described herein.

Nucleotide sequences corresponding to the amino acid sequences described herein, to be used as probes or primers for the isolation of nucleic acids or as query sequences for database searches, can be obtained by “back-translation” from the amino acid sequences. The well-known polymerase chain reaction (PCR) procedure can be employed to isolate and amplify a DNA sequence encoding antibody heavy chains and Fc-fusion proteins. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The variants according to the invention are ordinarily prepared by site specific mutagenesis of nucleotides in the DNA encoding the heavy chain or Fc-fusion protein, using cassette or PCR mutagenesis or other techniques well known in the art, to produce DNA encoding the variant, and thereafter expressing the recombinant DNA in cell culture as outlined herein. However, heavy chain and Fc-fusion proteins may be prepared by in vitro synthesis using established techniques. The variants typically exhibit the same qualitative biological activity as the naturally occurring analogue although variants can also be selected which have modified characteristics as will be more fully outlined below.

As will be appreciated by those in the art, due to the degeneracy of the genetic code, an extremely large number of nucleic acids may be made, all of which encode heavy chains and Fc-fusion proteins of the present invention. Thus, having identified a particular amino acid sequence, those skilled in the art could make any number of different nucleic acids, by simply modifying the sequence of one or more codons in a way which does not change the amino acid sequence of the encoded protein.

The present invention also provides expression systems and constructs in the form of plasmids, expression vectors, transcription or expression cassettes which comprise at least one polynucleotide as above. In addition, the invention provides host cells comprising such expression systems or constructs.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the heavy chain or Fc-fusion protein coding sequence; the oligonucleotide sequence encodes polyHis (such as hexaHis (SEQ ID NO: 58)), or another “tag” such as FLAG, HA (hemaglutinin influenza virus), or myc, for which commercially available antibodies exist. This tag is typically fused to the polypeptide upon expression of the polypeptide, and can serve as a means for affinity purification or detection of the protein from the host cell. Affinity purification can be accomplished, for example, by column chromatography using antibodies against the tag as an affinity matrix. Optionally, the tag can subsequently be removed from the purified heavy chain or Fc-fusion proteins by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antibody heavy chain or Fc-fusion protein. As a result, increased quantities of a polypeptide such as a heavy chain or Fc-fusion protein are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors of the invention will typically contain a promoter that is recognized by the host organism and operably linked to the molecule encoding the heavy chain or Fc-fusion protein. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe gene to which they are operably linked, that is, with little or no control over gene expression. A large number of promoters, recognized by a variety of potential host cells, are well known.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78:1444-1445); promoter and regulatory sequences from the metallothionine gene Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315:115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7:1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5:1639-1648; Hammer et al., 1987, Science 253:53-58); the alpha 1-antitrypsin gene control region that is active in liver (Kelsey et al., 1987, Genes and Devel. 1:161-171); the beta-globin gene control region that is active in myeloid cells (Mogram et al., 1985, Nature 315:338-340; Kollias et al., 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234:1372-1378).

An enhancer sequence may be inserted into the vector to increase transcription by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody or Fc-fusion protein. The choice of signal peptide or leader depends on the type of host cells in which the protein is to be produced, and a heterologous signal sequence can replace the native signal sequence. Examples of signal peptides that are functional in mammalian host cells include the following: the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The vector may contain one or more elements that facilitate expression when the vector is integrated into the host cell genome. Examples include an EASE element (Aldrich et al. 2003 Biotechnol Prog. 19:1433-38) and a matrix attachment region (MAR). MARs mediate structural organization of the chromatin and may insulate the integrated vector from “position” effect. Thus, MARs are particularly useful when the vector is used to create stable transfectants. A number of natural and synthetic MAR-containing nucleic acids are known in the art, e.g., U.S. Pat. Nos. 6,239,328; 7,326,567; 6,177,612; 6,388,066; 6,245,974; 7,259,010; 6,037,525; 7,422,874; 7,129,062.

Expression vectors of the invention may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding a heavy chain or Fc-fusion protein has been inserted into the proper site of the vector, the completed vector may be inserted into a suitable host cell for amplification and/or polypeptide expression. The transformation of an expression vector into a selected host cell may be accomplished by well known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

A host cell, when cultured under appropriate conditions, synthesizes a heavy chain or Fc-fusion protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. A host cell may be eukaryotic or prokaryotic.

Mammalian cell lines available as hosts for expression are well known in the art and include, but are not limited to, immortalized cell lines available from the American Type Culture Collection (ATCC) and any cell lines used in an expression system known in the art can be used to make the recombinant polypeptides of the invention. In general, host cells are transformed with a recombinant expression vector that comprises DNA encoding a desired heavy chain or Fc-fusion. Among the host cells that may be employed are prokaryotes, yeast or higher eukaryotic cells. Prokaryotes include gram negative or gram positive organisms, for example E. coli or bacilli. Higher eukaryotic cells include insect cells and established cell lines of mammalian origin. Examples of suitable mammalian host cell lines include the COS-7 line of monkey kidney cells (ATCC CRL 1651) (Gluzman et al., 1981, Cell 23:175), L cells, 293 cells, C127 cells, 3T3 cells (ATCC CCL 163), Chinese hamster ovary (CHO) cells, or their derivatives such as Veggie CHO and related cell lines which grow in serum-free media (Rasmussen et al., 1998, Cytotechnology 28: 31), HeLa cells, BHK (ATCC CRL 10) cell lines, and the CV)/EBNA cell line derived from the African green monkey kidney cell line CVI (ATCC CCL 70) as described by McMahan et al., 1991, EMBO J. 10: 2821, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays.

Alternatively, it is possible to produce the polypeptide in lower eukaryotes such as yeast or in prokaryotes such as bacteria. Suitable yeasts include Saccharomyces cerevisiae, Schizosaccharomyces pombe, Kluyveromyces strains, Candida, or any yeast strain capable of expressing heterologous polypeptides. Suitable bacterial strains include Escherichia coli, Bacillus subtilis, Salmonella typhimurium, or any bacterial strain capable of expressing heterologous polypeptides. If the polypeptide is made in yeast or bacteria, it may be desirable to modify the polypeptide produced therein, for example by phosphorylation or glycosylation of the appropriate sites, in order to obtain the functional polypeptide. Such covalent attachments can be accomplished using known chemical or enzymatic methods.

The polypeptide can also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). Cell-free translation systems could also be employed to produce polypeptides using RNAs derived from nucleic acid constructs disclosed herein. Appropriate cloning and expression vectors for use with bacterial, fungal, yeast, and mammalian cellular hosts are described by Pouwels et al. (Cloning Vectors: A Laboratory Manual, Elsevier, New York, 1985). A host cell that comprises an isolated nucleic acid of the invention, preferably operably linked to at least one expression control sequence, is a “recombinant host cell”.

### Pharmaceutical Compositions

The improved stability and reduced aggregation characteristics of the polypeptides of the invention renders them particularly useful for formulation into pharmaceutical compositions. Such compositions comprise one or more additional components such as a physiologically acceptable carrier, excipient or diluent. Optionally, the composition additionally comprises one or more physiologically active agents, for example, as described below. In various particular embodiments, the composition comprises one, two, three, four, five, or six physiologically active agents in addition to one or more antibody and/or Fc-fusion protein of the present invention.

In one embodiment, the pharmaceutical composition comprises an antibody and/or Fc-fusion protein of the invention together with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16th Ed. (1980) and 20th Ed. (2000), Mack Publishing Company, Easton, Pa.

Kits for use by medical practitioners are provided including one or more antibody and/or Fc-fusion proteins of the invention and a label or other instructions for use in treating any of the conditions discussed herein. In one embodiment, the kit includes a sterile preparation of one or more antibody and/or Fc-fusion protein, which may be in the form of a composition as disclosed above, and may be in one or more vials.

Dosages and the frequency of administration may vary according to such factors as the route of administration, the particular antibody and/or Fc-fusion protein employed, the nature and severity of the disease to be treated, whether the condition is acute or chronic, and the size and general condition of the subject. Appropriate dosages can be determined by procedures known in the pertinent art, e.g. in clinical trials that may involve dose escalation studies.

An antibody and/or Fc-fusion protein of the invention may be administered, for example, once or more than once, e.g., at regular intervals over a period of time. In particular embodiments, an antibody and/or Fc-fusion protein is administered over a period of at least once a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the antibody and/or Fc-fusion protein is administered until the patient manifests a medically relevant degree of improvement over baseline for the chosen indicator or indicators.

As is understood in the pertinent field, pharmaceutical compositions comprising the antibody and/or Fc-fusion protein of the invention are administered to a subject in a manner appropriate to the indication. Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of the antibody and/or Fc-fusion protein in aerosol form, and the like. Other alternatives include oral preparations including pills, syrups, or lozenges.

### DEFINITIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings: The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

Polynucleotide and polypeptide sequences are indicated using standard one- or three-letter abbreviations. Unless otherwise indicated, polypeptide sequences have their amino termini at the left and their carboxy termini at the right, and single-stranded nucleic acid sequences, and the top strand of double-stranded nucleic acid sequences, have their 5′ termini at the left and their 3′ termini at the right. A particular polypeptide or polynucleotide sequence also can be described by explaining how it differs from a reference sequence.

The terms “peptide” “polypeptide” and “protein” each refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric.

The term “polypeptide fragment” as used herein refers to a polypeptide that has an amino-terminal and/or carboxy-terminal deletion as compared to a corresponding full-length protein. Fragments can be, for example, at least 5, 6, 7, 8, 9, 10, 11, 12, 13, 14, 15, 20, 50, 70, 80, 90, 100, 150, 200, 250, 300, 350, or 400 amino acids in length. Fragments can also be, for example, at most 1,000, 750, 500, 250, 200, 175, 150, 125, 100, 90, 80, 70, 60, 50, 40, 30, 20, 15, 14, 13, 12, 11, or 10 amino acids in length. A fragment can further comprise, at either or both of its ends, one or more additional amino acids, for example, a sequence of amino acids from a different naturally-occurring protein or an artificial amino acid sequence.

Polypeptides of the invention include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties. Analogs include muteins of a polypeptide. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” is one that does not substantially change the structural characteristics of the parent sequence (e.g., a replacement amino acid should not tend to break a helix that occurs in the parent sequence, or disrupt other types of secondary structure that characterize the parent sequence or are necessary for its functionality). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

A “variant” of a polypeptide comprises an amino acid sequence wherein one or more amino acid residues are inserted into, deleted from and/or substituted into the amino acid sequence relative to another polypeptide sequence. Variants of the invention include those comprising variant CH2 or CH3 domains. In certain embodiments, a variant comprises one or more mutations that when present in an Fc molecule increase affinity for the polypeptide to one or more FcγRs. Such variants demonstrate enhanced antibody-dependent cell-mediated cytotoxicity. Examples of variants providing such are described in U.S. Pat. No. 7,317,091.

Other variants include those that decrease the ability of CH3-domain containing polypeptides to homodimerize, while increasing the ability to heterodimerize. Examples of such Fc variants are described in U.S. Pat. Nos. 5,731,168 and 7,183,076. Further examples are described in the co-owned U.S. Provisional Applications 61/019,569, filed Jan. 7, 2008, and 61/120,305, filed Dec. 5, 2008 (both incorporated by reference in their entirety).

A “derivative” of a polypeptide is a polypeptide (e.g., an antibody) that has been chemically modified, e.g., via conjugation to another chemical moiety such as, for example, polyethylene glycol, a cytotoxic agent, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described herein.

The CH3 domain-containing polypeptide can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991. Intact antibodies include polyclonal, monoclonal, chimeric, humanized or fully human having full length heavy and light chains.

An antibody may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes. One or more genes encoding the human heavy chains may be altered to contain a Ser362 mutation. When such mice are immunized with an antigen, the mice will produce human antibodies having a Ser364 mutation.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity.

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases.

Computerized comparison methods can be used to identify sequence motifs or predicted protein conformation domains that occur in other proteins of known structure and/or function.

Methods to identify protein sequences that fold into a known three-dimensional structure are known. See, e.g., Bowie et al., 1991, Science 253:164.

A “CDR grafted antibody” is an antibody comprising one or more CDRs derived from an antibody of a particular species or isotype and the framework of another antibody of the same or different species or isotype.

A “multi-specific antibody” is an antibody that recognizes more than one epitope on one or more antigens. A subclass of this type of antibody is a “bi-specific antibody”.

The “percent identity” of two polynucleotide or two polypeptide sequences is determined by comparing the sequences using the GAP computer program (a part of the GCG Wisconsin Package, version 10.3 (Accelrys, San Diego, Calif.)) using its default parameters.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In one embodiment, the nucleic acid molecules of the invention comprise a contiguous open reading frame encoding an antibody or an Fc-fusion, and a derivative, mutein, or variant thereof.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al, 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Exemplary host cells include Chinese hamster ovary (CHO) cell lines or their derivatives including CHO strain DXB-11, which is deficient in DHFR (see Urlaub et al, 1980, Proc. Natl. Acad. Sci. USA 77:4216-20), CHO cell lines which grow in serum-free media (see Rasmussen et al., 1998, Cytotechnology 28:31), CS-9 cells, a derivative of DXB-11 CHO cells, and AM-1/D cells (described in U.S. Pat. No. 6,210,924). Other CHO cells lines include CHO-K1 (ATCC# CCL-61), EM9 (ATCC# CRL-1861), and UV20 (ATCC# CRL-1862). Examples of other host cells include COS-7 line of monkey kidney cells (ATCC CRL 1651) (see Gluzman et al., 1981, Cell 23:175), L cells, C 127 cells, 3T3 cells (ATCC CCL 163), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell.

The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

### EXAMPLES

The following examples, including the experiments conducted and results achieved, are provided for illustrative purposes only and are not to be construed as limiting the present invention.

### Example 1

### Preparation of Cysteine Clamp Constructs

Peptide fusions with charged pair (delHinge) cysteine clamp Fc sequences were stably expressed in serum free, suspension adapted CHO-K1 cell line. Fc fusion molecules were cloned into a stable expression vector containing puromycin resistance while the Fc chains were cloned into a hygromycin containing expression vector (Selexis, Inc.). The plasmids were transfected at a 1:1 ratio using lipofectamine LTX and cells were selected 2 days post transfection in growth media containing 10 ug/mL puromycin and 600 ug/mLhygromycin. Media was exchanged 2 times per week during selection. When cells reached about 90% viability, they were scaled up for a fedbatch production run. Cells were seeded at 1e6/mL in a production media and fed on days 3, 6, and 8. The conditioned medium (CM) produced by the cells was harvested on day 10 and clarified. Endpoint viabilities typically were above 90%.

The Fc-fusions clarified, conditioned media was purified using a two-step chromatography procedure. Approximately 5 L of the CM was applied directly to a GE MabSelect SuRe column that had previously been equilibrated with Dulbecco's Phosphate Buffered Saline (PBS). The bound protein underwent three wash steps: first, 3 column volumes (CV) of PBS; next, 1 CV of 20 mM Tris, 100 mM sodium chloride, pH 7.4; and finally, 3 CV of 500 mM L-arginine, pH 7.5. These wash steps remove unbound or lightly bound media components and host cell impurities. The column was then re-equilibrated with 5 CV of 20 mM Tris, 100 mM sodium chloride at pH 7.4 which brings the UV absorbance back to baseline. The desired protein was eluted with 100 mM acetic acid at pH 3.6 and collected in bulk. The protein pool was quickly titrated to within a pH range of 5.0 to 5.5 with 1 M Tris-HCl, pH 9.2.

The pH adjusted protein pool was next loaded onto a GE SP Sepharose HP column that had been previously equilibrated with 20 mM MES at pH 6.0. The bound protein was then washed with 5 CV of equilibration buffer, and finally eluted over a 20 CV, 0 to 50% linear gradient from 0 to 400 mM sodium chloride in 20 mM MES at pH 6.0. Fractions were collected during the elution and analyzed by analytical size-exclusion chromatography (Superdex 200) to determine the appropriate fractions to pool for a homogeneous product. The SP HP chromatography removes product-related impurities such as free Fc, clipped species, and Fc-GDF15 multimers.

The SP HP pool was then buffer exchanged into a formulation buffer by dialysis. It was concentrated to approximately 15 mg/ml using the Sartorius Vivaspin 20 Ten kilo-Dalton molecular weight cut-off centrifugal device. Finally, it was sterile filtered and the resulting solution containing the purified Fc fusion molecules is stored at 5° C. Final products were assessed for identity and purity using mass spectral analysis, sodium dodecyl sulfate polyacrylamide electrophoresis and size exclusion high performance liquid chromatography.

### Example 2

### Analysis of Cysteine Clamp Constructs

Disulfide Bond Formation

Fc fusion proteins lacking the hinge region and having a L351C mutation were expressed and purified as described above.

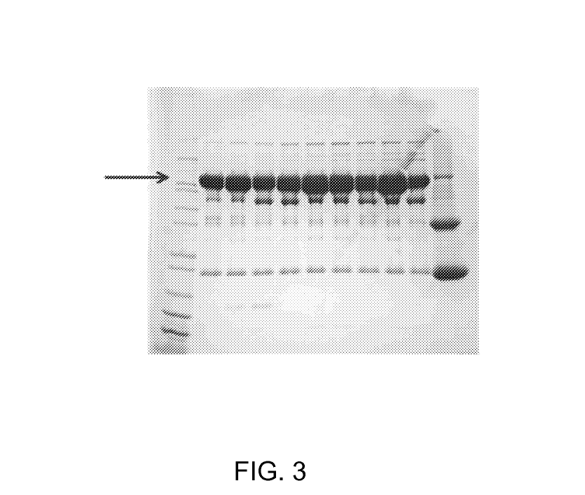

The samples were analyzed by SDS polyacrylamide gel electrophoresis. Constructs with different molecular weight have different mobility on the SDS-PAGE. This facilitates the identification of the associated chains. In FIG. 3, the last lane corresponds to reduced condition in which the disulfide bonds are broken and the other lanes correspond to non-reduced condition of various fractions from the purification process. The disulfides are kept intact under the non-reduced condition. The higher band observed under the non-reduced condition demonstrates the introduced covalent link via disulfide bond between the two Fc chains at the CH3 domain is indeed formed. And the last lane in FIG. 3 demonstrates that upon reduced condition the engineered disulfide breaks as expected leading to double bands.

Pharmacokinetics Analysis

Six Fc fusion protein constructs lacking the hinge region (A-F) were compared.

A. Fc fusion lacking hinge and without a linker between the therapeutic peptide and the Fc.

B. Same as A except with a variation within the therapeutic peptide.

C. Same as B except a non-glycosylated linker connects the Fc to the therapeutic peptide.

D. Same as C except with a different linker.

E. Same as A except one Fc chain comprises a Y349C substitution and the other comprises an S354C substitution.

F. Same as B except one Fc chain comprises a Y349C substitution and the other comprises an S354C substitution.

Test articles were administered intravenously via the tail vein to male diet-induced obese CD-1 mice (n=3 per test article) at a dose of 1 mg/kg. Serial blood samples (50 uL per time point) were collected from each animal at the following time points: 1, 4, 8, 24, 72, 168, 240, and 336 hours post-dose. Test article concentrations in serum samples were quantified using a sandwich ELISA that utilizes anti-test article antibodies for capture and detection. The lower limit of quantitation for the assay was 313 ug/L. Concentration-time profiles were plotted and non-compartmental analysis was performed to calculate PK parameters using Watson.

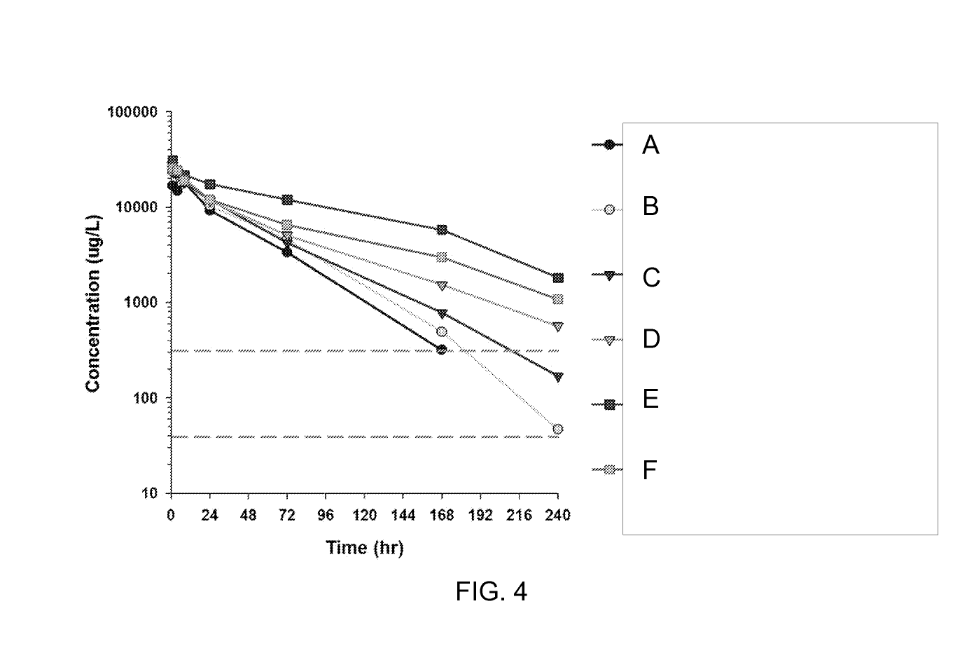

As seen in FIG. 4, of the six variants tested, the cysteine clamp variants, E and F, had the lowest overall systemic clearance and correspondingly, highest exposure (expressed as area under the concentration-time curve, AUC). E demonstrated >3 fold higher AUC than the non cysteine clamp version A while F demonstrated a 1.6 fold improvement in AUC over B. Thus, the pharmacokinetics of Fc fusion proteins lacking a hinge region are significantly improved by the introduction of a disulfide bond into the CH3 interface.

1. A polypeptide comprising an antibody Fc region, said Fc region comprising a deletion or substitution of one or more cysteines of the hinge region and substitution of one or more CH3-interface amino acids with a sulfhydryl containing residue.

2. The polypeptide of claim 1, wherein the Fc lacks a cysteine-containing portion of the hinge region.

3. The polypeptide of claim 2, wherein the Fc lacks the hinge region.

4. The polypeptide of claim 1, wherein all cysteines within the hinge region are substituted for another amino acid.

5. The polypeptide of claim 1, wherein the CH3-interface amino acid substituted with a sulfhydryl containing residue is Y349, L351, S354, T394, or Y407.

6. The polypeptide of claim 5, wherein Y349 is substituted with cysteine (Y349C).

7. The polypeptide of claim 5, wherein L351 is substituted with cysteine (L351C).

8. The polypeptide of claim 5, wherein S354 is substituted with cysteine (S354C).

9. The polypeptide of claim 5, wherein T394 is substituted with cysteine (T394C).

10. The polypeptide of claim 5, wherein Y407 is substituted with cysteine (Y407C).

11. The polypeptide of claim 1, wherein the Fc comprises a CH2 region comprising one or more amino acid substitutions.

12. The polypeptide of claim 1, wherein the CH3 region further comprises one or more additional amino acids substitutions.

13. The polypeptide of claim 1, wherein one or more amino acids of the C-terminus of the Fc are deleted.

14. The polypeptide of claim 13, wherein three, two, or one amino acid of the C-terminus of the Fc is deleted.

15. The polypeptide of claim 14, wherein the terminal amino acid of the C-terminus of the Fc is deleted.

16. The polypeptide of claim 1, wherein the polypeptide comprises an antibody heavy chain.

17. The polypeptide of claim 1, wherein the polypeptide comprises an Fc fusion protein.

18. A nucleic acid encoding a polypeptide of claim 1.

19. An expression vector comprising the nucleic acid of claim 18 operably linked to a promoter.

20. A host cell comprising the expression vector of claim 19.

21. Method of making a polypeptide, said method comprising
a) culturing the host cell of claim 20 under conditions in which the promoter is active in said host cell; and
b) isolating the polypeptide from the culture.

22. A pharmaceutical composition comprising the polypeptide of claim 1.


SEQUENCE LISTING (58 sequences)

SEQ ID NO: 1 - protein, 330 aa, Homo sapiens [sequence omitted]

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 6 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 7 - protein, 428 aa, Homo sapiens
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 9 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 17 aa, Homo sapiens
DKTHTCPPCPAPELLGG

SEQ ID NO: 11 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, Homo sapiens
RKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 233 aa, Homo sapiens
LKTPLGDTTHTCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 228 aa, Homo sapiens
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 227 aa, Mus sp.
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 16 - protein, 237 aa, Mus sp.
DKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 239 aa, Mus sp.
EPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 18 - protein, 238 aa, Mus sp.
EPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 19 - protein, 233 aa, Mus sp.
EPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 20 - protein, 233 aa, Ovis aries
EPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 21 - protein, 228 aa, Ovis aries
GISSDYSKCSKPPCVSRPSVFIFPPKPKDSLMITGTPEVTCVVVDVQGDPEVQFSWFVDNVEVRTARTKPREEQFNSTFRVVSALPIQHDHWTGGKEFKCKVHSKGLPAPIVRTISRAKGQAREPQVYVLAPPQEELSKSTLSVTCLVTGFYPDYIAVEWQRARQPESEDKYGTTTSQLDADGSYFLYSRLRVDKSSWQRGDTYACVVMHEALHNHYTQKSISKPPGK

SEQ ID NO: 22 - protein, 232 aa, Bos taurus
DPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 23 - protein, 230 aa, Bos taurus
GVSSDCSKPNNQHCCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSVTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 24 - protein, 232 aa, Bos taurus
KSEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSKLRVNKSSWQEGDHYTCAVMHEALRNHYKEKSISRSPGK

SEQ ID NO: 25 - protein, 229 aa, Rattus sp.
VPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 26 - protein, 225 aa, Rattus sp.
VPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 27 - protein, 238 aa, Rattus sp.
ERRNGGIGHKCPTCPTCHKCPVPELLGGPSVFIFPPKPKDILLISQNAKVTCVVVDVSEEEPDVQFSWFVNNVEVHTAQTQPREEQYNSTFRVVSALPIQHQDWMSGKEFKCKVNNKALPSPIEKTISKPKGLVRKPQVYVMGPPTEQLTEQTVSLTCLTSGFLPNDIGVEWTSNGHIEKNYKNTEPVMDSDGSFFMYSKLNVERSRWDSRAPFVCSVVHEGLHNHHVEKSISRPPGK

SEQ ID NO: 28 - protein, 228 aa, Oryctolagus cuniculus
APSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 29 - protein, 239 aa, Equus caballus
EPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDEDSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 30 - protein, 232 aa, Equus caballus
ARVTPVCSLCRGRYPHPIGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 31 - protein, 256 aa, Equus caballus
EPVLPKPTTPAPTVPLTTTVPVETTTPPCPCECPKCPAPELLGGPSVFIFPPKPKDVLMITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 32 - protein, 230 aa, Equus caballus
VIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRAELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 33 - protein, 230 aa, Equus caballus
VVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 34 - protein, 231 aa, Equus caballus
VIKEPCCCPKCPDSKFLGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 35 - protein, 235 aa, Macaca fascicularis
EIKTCGGGSKPPTCPPCPAPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWRQGNVFSCSVMHEALHNHYTQKSLSLSPGK

SEQ ID NO: 36 - protein, 235 aa, Macaca mulatta
EIKTCGGGSKPPTCPPCTSPELLGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCLVKGFYPSDIVVEWESSGQPENTYKTTPPVLDSDGSYFLYSKLTVDKSRWQQGNVFSCSVMHEALHNHYTQKSLSLSPGK

SEQ ID NO: 37 - protein, 228 aa, Macaca mulatta
GLPCRSTCPPCPAELLGGPSVFLFPPKPKDTLMISRTPEVTCVVVDVSQEEPDVKFNWYVDGVEVHNAQTKPREEQFNSTYRVVSVLTVTHQDWLNGKEYTCKVSNKALPAPKQKTVSKTKGQPREPQVYTLPPPRKELTKNQVSLTCLVKGFYPSDIVVEWASNGQPENTYKTTPPVLDSDGSYFLYSKLTVDKSRWQQGNTFSCSVMHEALHNHYTQKSLSLSPGK

SEQ ID NO: 38 - protein, 232 aa, Macaca mulatta
EFTPPCGDTTPPCPPCPAPELLGGPSVFLFPPKPKDTLMISRTPEVTCVVVDVSQEDPEVQFNWYVDGAEVHHAQTKPREEQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYFLYSKLTVDKSRWQQGNTFSCSVMHEALHNHYTQKSLSVSP

SEQ ID NO: 39 - protein, 230 aa, Sus scrofa
GIHQPQTCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

SEQ ID NO: 40 - protein, 230 aa, Sus scrofa
GTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 41 - protein, 230 aa, Sus scrofa; identical to SEQ ID NO: 40

SEQ ID NO: 42 - protein, 230 aa, Sus scrofa
GTKTKPPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 43 - protein, 230 aa, Sus scrofa
GTKTKPPCPICPACEGPGPSAFIFPPKPKDTLMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYTQKSIFKTPGK

SEQ ID NO: 44 - protein, 226 aa, Sus scrofa
GRPCPICPACEGPGPSAFIFPPKPKDTFMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKLSVTCLITGFYPPDIDVEWQRNGQPEPEGNYRTTPPQQDVDGTYFLYSKLAVDKASWQRGDPFQCAVMHEALHNHYTQKSIFKTPGN

SEQ ID NO: 45 - protein, 5 aa, Artificial Sequence
  Description of Artificial Sequence Synthetic peptide
GGGGS

SEQ ID NO: 46 - protein, 5 aa, Artificial Sequence
  Description of Artificial Sequence Synthetic peptide
GGNGT

SEQ ID NO: 47 - protein, 5 aa, Artificial Sequence
  Description of Artificial Sequence Synthetic peptide
YGNGT

SEQ ID NO: 48 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 49 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 50 - protein, 332 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGGGGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 51 - protein, 351 aa, Artificial Sequence
  Description of Artificial Sequence Synthetic polypeptide
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGGGGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 52 - protein, 236 aa, Artificial Sequence
  Description of Artificial Sequence Synthetic polypeptide
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 53 - protein, 216 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRKEMTKNQVSLTCLVKGFYPSDIAVEWESNGQPENNYKTTPPVLKSDGSFFLYSKLTVDKSRWQQGNVFSCSVMHEALHNHYTQKSLSLSPG

SEQ ID NO: 54 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 55 - protein, 332 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRKEMTKNQVSLTCLVKGFYPSDIAVEWESNGQPENNYKTTPPVLKSDGSFFLYSKLTVDKSRWQQGNVFSCSVMHEALHNHYTQKSLSLSPGGGGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 56 - protein, 351 aa, Artificial Sequence
  Description of Artificial Sequence Synthetic polypeptide
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRKEMTKNQVSLTCLVKGFYPSDIAVEWESNGQPENNYKTTPPVLKSDGSFFLYSKLTVDKSRWQQGNVFSCSVMHEALHNHYTQKSLSLSPGGGGGARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 57 - protein, 236 aa, Artificial Sequence
  Description of Artificial Sequence Synthetic polypeptide
MEWSWVFLFFLSVTTGVHSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 58 - protein, 6 aa, Artificial Sequence
  Description of Artificial Sequence Synthetic 6xHis tag
HHHHHH